(19) United States
(12) Patent Application Publication
(10) Pub. No.: US 2014/0371316 A1
(43) Pub. Date: Dec. 18, 2014

(54) DERIVATIVES OF PHENOXYISOBUTYRIC ACID

(71) Applicants: Jill S. FABRICANT, (US); Iraj LALEZARI, (US)

(72) Inventors: Iraj Lalezari, Louisville, CO (US); Jill S. Fabricant, Corona Del Mar, CA (US)

(21) Appl. No.: 14/360,252

(22) PCT Filed: Nov. 23, 2012
(86) PCT No.: PCT/US12/66443

§ 371 Date: May 22, 2014

Related U.S. Application Data
(60) Provisional application No. 61/563,350, filed on Nov. 23, 2011.
(60) Provisional application No. 61/742,737, filed on Aug. 17, 2012.

(51) Int. Cl. C07D 265/30; C07D 213/55; C07D 235/18; A61Q 19/08; C07C 217/92; A61K 8/41; A61K 8/49; C07D 211/08; C07C 237/40

(52) U.S. Cl. CPC C07D 265/30; C07D 211/08; C07D 213/55; C07D 235/18; C07C 237/40; C07C 217/92; A61K 8/411; A61K 8/4926; A61K 8/49; A61K 8/4946; A61Q 19/08
USPC 514/563; 514/237.8; 514/239.5; 514/331; 514/357; 514/394; 514/566; 514/567; 544/171; 546/235; 546/335; 548/309.7; 562/450; 562/457

(57) ABSTRACT

The present invention provides a process for the synthesis of substituted phenoxymethylpropiomc acid and related compounds. The compounds are useful for inhibiting the formation of AGEs (Advanced Glycation End Products).

The present invention relates generally to the synthesis and production of novel derivatives of phenoxyisobutyric acid that are useful in pharmaceutical and cosmetic applications. One use of the disclosed compounds is as anti AGE (Advanced Glycation Products) compound for the treatment of diabetes.

It is known in the art that elevated concentration of reducing sugars in the blood and in the intracellular environment results in the nonenzymatic formation of glycation and dehydration condensation complexes known as advanced glycation end-products or aminaglycation end products (AGEs). Nonenzymatic glycation is a complex series of reactions between reducing sugars and amino groups of proteins, lipids, and DNA. These complex products form on free amino groups on proteins, on lipids and on DNA (Bucala and Cerami, 1992; Bucala et al., 1993; Bucala et al., 1984). This phenomenon is called “browning” or a “Maillard” reaction and was discovered early in the last century by the food industry (Maillard, 1916). The reaction is initiated with the reversible formation of Schiff's base which undergoes rearrangement to form a stable Amadori product. Both Schiff's base and Amadori product further undergo a series of reactions through dicarbonyl intermediates to form AGEs. The significance of a similar process in biology became evident only after the discovery of the glycosylated hemoglobins and their increased presence in diabetic patients (Rahbar, 1968; Rahbar et al., 1969). In human diabetic patients and in animal models of diabetes, these nonenzymatic reactions are accelerated and cause increased AGE formation and increased glycation of long-lived proteins such as collagen, fibronectin, tubulin, lens crystallin, myelin, laminin and actin, in addition to hemoglobin and albumin, and also of LDL associated lipids and apoprotein. Moreover, brown pigments with spectral and fluorescent properties similar to those of late-stage Maillard products have also been found in vivo in association with several long-lived proteins such as crystalline lens proteins and collagen from aged individuals. An age-related linear increase in pigments was observed in human dura collagen between the ages of 20 to 90 years. AGE modified proteins increase slowly with aging and are thought to contribute to normal tissue remodeling. Their level increases markedly in diabetic patients as a result of sustained high blood sugar levels and lead to tissue damage through a variety of mechanisms including alteration of tissue protein structure and function, stimulation of cellular responses through AGE specific receptors or the generation of reactive oxygen species (ROS) (for a recent review see Boel et al., 1995). The structural and functional integrity of the affected molecules, which often have major roles in cellular functions, become disturbed by these modifications, with severe consequences on affected organs such as kidney, eye, nerve, and micro-vascular functions (Silbiger et al., 1993; Brownlee et al., 1985).

Structural changes on macromolecules by AGEs are known to accumulate under normal circumstances with increasing age. This accumulation is severely accelerated by diabetes and is strongly associated with hyperglycemia. For example, formation of AGE on protein in the subendothelial basement membrane causes extensive cross-link formation which leads to severe structural and functional changes in protein/protein and protein/cell interaction in the vascular wall (Haitoglou et al., 1992; Airaksinen et al., 1993).

Enhanced formation and accumulation of advanced glycation end products (AGEs) have been implicated as a major pathogenesis process leading to diabetic complications, normal aging, atherosclerosis and Alzheimer's disease. This process is accelerated by diabetes and has been postulated to contribute to the development of a range of diabetic complications including nephropathy (Nicholls and Mandel, 1989), retinopathy (Hammes et al., 1991) and neuropathy (Cameron et al., 1992). Particularly, tissue damage to the kidney by AGEs leads to progressive decline in renal function, end-stage renal disease (ESRD) (Makita et al., 1994), and accumulation of low-molecular-weight (LMW) AGE peptides (glycotoxins) (Koschinsky et al., 1997) in the serum of patients with ESRD (Makita et al., 1991). These low molecular weight (LMW)-AGEs can readily form new crosslinks with plasma or tissue components, e.g., low density lipoprotein (LDL) (Bucala et al., 1994) or collagen (Miyata et al., 1993) and accelerate the progression of tissue damage and morbidity in diabetics.

Direct evidence indicating the contribution of AGEs in the progression of diabetic complications in different lesions of the kidneys, the rat lens and in atherosclerosis has been reported (Vlassara et al., 1994; Vlassara et al., 1995; Horie et al., 1997; Matsumoto et al., 1997; Soulis-Liparota et al., 1991; Bucala and Vlassara, 1997; Bucala and Rahbar, 1998; Park et al., 1998). Indeed, the infusion of pre-formed AGEs into healthy rats induces glomerular hypertrophy and mesangial sclerosis, gene expression of matrix proteins and production of growth factors (Brownlee et al., 1991; Vlassara et al., 1995). Several lines of evidence indicate that the increase in reactive carbonyl intermediates (methylglyoxal, glycolaldehyde, glyoxal, 3-deoxyglucosone, malondialdehyde and hydroxynonenal) is the consequence of hyperglycemia in diabetes. “Carbonyl stress” leads to increased modification of proteins and lipids, followed by oxidant stress and tissue damage (Baynes and Thorpe, 1999; Onorato et al., 1998; McLellan et al., 1994). Further studies have revealed that aminoguanidine (AG), an inhibitor of AGE formation, ameliorates tissue impairment of glomeruli and reduces albuminuria in induced diabetic rats (Soulis-Liparota et al., 1991; Itakura et al., 1991). In humans, decreased levels of hemoglobin (Hb)-AGE (Makita et al., 1992) concomitant with amelioration of kidney function as the result of aminoguanidine therapy in diabetic patients, provides more evidence for the importance of AGEs in the pathogenesis of diabetic complications (Bucala and Vlassara, 1997).

The global prevalence of diabetes mellitus, in particular in the United States, afflicting millions of individuals with significant increases of morbidity and mortality, together with the great financial burden for the treatment of diabetic complications in this country, are major incentives to search for and develop drugs with a potential for preventing or treating complications of the disease. So far the mechanisms of hyperglycemia-induced tissue damage in diabetes are not well understood. However, four pathogenic mechanisms have been proposed, including increased polyol pathway activity, activation of specific protein kinase C (PKC) isoforms, formation and accumulation of advanced glycation endproducts, and increased generation of reactive oxygen species (ROS) (Kennedy and Lyons, 1997). Most recent immunohistochemical studies on different tissues from kidneys obtained from ESRD patients (Horie et al., 1997) and diabetic rat lenses (Matsumoto et al., 1997), by using specific antibodies against carboxymethyllysine (CML), pentosidine, the two known glycoxidation products and pyrraline, have localized these AGE components in different lesions of the kidneys and the rat lens, and have provided more evidence in favor of protein-AGE formation in close association with generation of ROS to be major factors in causing permanent and irreversible modification of tissue proteins. Therefore, inhibitors of AGE formation and antioxidants hold promise as effective means of prevention and treatment of diabetic complications.

The Diabetic Control and Complications Trial (DCCT), has identified hyperglycemia as the main risk factor for the development of diabetic complications (The Diabetes Control and Complications Trial Research Group, 1993). Compelling evidence identifies the formation of advanced glycation endproducts as the major pathogenic link between hyperglycemia and the long-term complications of diabetes (Makita et al., 1994; Koschinsky et al., 1997; Makita et al., 1993; Bucala et al., 1994; Bailey et al., 1998).

The reactions between reducing sugars and amino groups of proteins, lipids and DNA undergo a series of reactions through dicarbonyl intermediates to generate advanced glycation endproducts (Bucala and Cerami, 1992; Bucala et al., 1993; Bucala et al., 1984).

In human diabetic patients and in animal models of diabetes, AGE formation and accumulation of long-lived structural proteins and lipoproteins have been reported. Most recent reports indicate that glycation inactivates metabolic enzymes (Yan and Harding, 1999; Kato et al., 2000; Verbeke et al., 2000; O'Harte et al., 2000). The glycation-induced change of immunoglobin G is of particular interest. Reports of glycation of the Fab fragment of IgG in diabetic patients suggest that immune deficiency observed in these patients may be explained by this phenomenon (Lapolla et al., 2000). Furthermore, an association between IgM response to IgG damaged by glycation and disease activity in rheumatoid arthritis has been reported (Lucey et al., 2000). Also, impairment of high-density lipoprotein function by glycation has been described (Hedrick et al., 2000).

Methylglyoxal (MG) has recently received considerable attention as a common mediator and the most reactive dicarbonyl to form AGEs (Phillips and Thomalley, 1993; Beisswenger et al., 1998). It is also a source of reactive oxygen species (ROS) (free radicals) generation in the course of glycation reactions (Yim et al., 1995).

Nature has devised several humoral and cellular defense mechanisms to protect tissues from the deleterious effects of “carbonyl stress” and accumulation of AGEs, e.g., the glyoxylase systems (I and II) and aldose reductase catalyze the detoxification of MG to D-lactate (McLellan et al., 1994). Amadoriases are also a novel class of enzymes found in Aspergillus which catalyze the deglycation of Amadori products (Takahashi et al., 1997). Furthermore, several AGE-receptors have been characterized, on the surface membranes of monocytes and on macrophage, endothelial, mesangial and hepatic cells. One of these receptors, RAGE, a member of the immunoglobulin superfamily, has been found to have a wide tissue distribution (Schmidt et al., 1994; Yan et al., 1997). The discovery of various natural defense mechanisms against glycation and AGE formation suggests an important role of AGEs in the pathogenesis of vascular and peripheral nerve damage in diabetes. MG binds to and irreversibly modifies arginine and lysine residues in proteins. MG modified proteins have been shown to be ligands for the AGE receptor (Westwood et al., 1997) indicating that MG modified proteins are analogous (Schalkwijk et al., 1998) to those found in AGEs. Furthermore, glycolaldehyde, a reactive intermediate in AGE formation, generates an active ligand for macrophage scavenger receptor (Nagai et al., 2000). The effects of MG on LDL have been characterized in vivo and in vitro (Bucala et al., 1993).

Lipid peroxidation of polyunsaturated fatty acids (PUFA), such as arachidonate, also yields carbonyl compounds; some are identical to those formed from carbohydrates (Al-Abed et al., 1996), such as MG and GO, and others are characteristic of lipids, such as malondialdehyde (MDA) and 4-hydroxynonenal (HNE) (Requena et al., 1997). The latter two carbonyl compounds produce lipoxidation products (Al-Abed et al., 1996; Requena et al., 1997). A recent report emphasizes the importance of lipid-derived MDA in the cross-linking of modified collagen and in diabetes mellitus (Slatter et al., 2000). A number of AGE compounds, both fluorophores and nonfluorescent, are involved in crosslinking proteins and have been characterized (Baynes and Thorpe, 1999). In addition to glucose derived AGE-protein crosslinks, AGE crosslinking also occurs between tissue proteins and AGE-containing peptide fragments formed from AGE-protein digestion and turnover. These reactive AGE-peptides, now called glycotoxins, are normally cleared by the kidneys. In diabetic patients, these glycotoxins react with the serum proteins and are a source for widespread tissue damage (He et al., 1999).

However, detailed information on the chemical nature of the crosslink structures remain unknown. The crosslinking structures characterized to date, on the basis of chemical and spectroscopic analyses, constitute only a small fraction of the AGE crosslinks which occur in vivo, with the major crosslinking structure(s) still unknown. Most recently, a novel acid-labile AGE-structure, N-omega-carboxymethylarginine (CMA), has been identified by enzymatic hydrolysis of collagen. Its concentration was found to be 100 times greater than the concentration of pentosidine (Iijima et al., 2000) and it is assumed to be a major AGE crosslinking structure.

In addition to aging and diabetes, the formation of AGEs has been linked with several other pathological conditions. IgM anti-IgG-AGE appears to be associated with clinical measurements of rheumatoid arthritis activity (Lucey et al., 2000). A correlation between AGEs and rheumatoid arthritis was also made in North American Indians (Newkirk et al., 1998). AGEs are present in brain plaques in Alzheimer's disease and the presence of AGEs may help promote the development of Alzheimer's disease (Durany et al., 1999; Munch et al., 1998; Munch et al., 1997). Uremic patients have elevated levels of serum AGES compared to age-matched controls (Odani et al., 1999; Dawnay and Millar, 1998). AGEs have also been correlated with neurotoxicity (Kikuchi et al., 1999). AGE proteins have been associated with atherosclerosis in mice (Sano et al., 1999) and with atherosclerosis in persons undergoing hemodialysis (Takayama et al., 1998). A study in which aminoguanidine was fed to rabbits showed that increasing amounts of aminoguanidine led to reduced plaque formation in the aorta thus suggesting that advanced glycation may participate in atherogenesis and raising the possibility that inhibitors of advanced glycation may retard the process (Panagiotopoulos et al., 1998). Significant deposition of N(epsilon)-carboxymethyl lysine (CML), an advanced glycation endproduct, is seen in astrocytic hyaline inclusions in persons with familial amyotrophic lateral sclerosis but is not seen in normal control samples (Kato et al., 1999; Shibata et al., 1999). Cigarette smoking has also been linked to increased accumulation of AGEs on plasma low density lipoprotein, structural proteins in the vascular wall, and the lens proteins of the eye, with some of these effects possibly leading to pathogenesis of atherosclerosis and other diseases associated with tobacco usage (Nicholl and Bucala, 1998). Finally, a study in which aminoguanidine was fed to rats showed that the treatment protected against progressive cardiovascular and renal decline (Li et al., 1996).

The mechanism of the inhibitory effects of aminoguanidine in the cascade of glycosylation events has been investigated. To date, the exact mechanism of AG-mediated inhibition of AGE formation is not completely known. Several lines of in vitro experiments resulted in contrasting conclusions. Briefly, elevated concentrations of reducing sugars cause reactions between carbohydrate carbonyl and protein amino groups leading to: 1. Reversible formation of Schiff's bases followed by 2. Amadori condensation/dehydration products such as 3-deoxyglucason (3-DG), a highly reactive dicarbonyl compound (Kato et al., 1990). 3. Irreversible and highly reactive advanced glycosylation endproducts. Examples of early Amadori products are ketoamines which undergo further condensation reactions to form late AGEs. A number of AGE products have been purified and characterized recently, each one constituting only minor fractions of the in vivo generated AGEs. Examples are pyrraline, pentosidine, carboxymethyl-lysine (CML), carboxyethyl-lysine (CEL), crossline, pyrrolopyridinium, methylglyoxal lysine dimer (MOLD), Arg-Lys imidazole, arginine pyridinium, cypentodine, piperidinedinone enol and alkyl, formyl, diglycosyl-pyrrole (Vlassara, 1994).

Analysis of glycation products formed in vitro on a synthetic peptide has demonstrated that aminoguanidine does not inhibit formation of early Amadori products (Edelstein and Brownlee, 1992). Similar conclusions were reached by analysis of glycation products formed on BSA (Requena et al., 1993). In both experiments AGE formation was strongly inhibited by AG as analyzed by fluorescence measurements and by mass spectral analysis. The mass spectral analysis, did not detect peptide complexes with molecular mass corresponding to an incorporation of AG in the complex. Detailed mechanistic studies using NMR, mass spectroscopy and X-ray diffraction have shown that aminoguanidine reacts with AGE precursor 3-DG to form 3-amino-5- and 3-amino-6-substituted triazines (Hirsch et al., 1992). In contrast, other experiments using labeled .sup.14C-AG with lens proteins suggest that AG becomes bound to the proteins and also reacts with the active aldose form of free sugars (Harding, 1990).

Several other potential drug candidates as AGE inhibitors have been reported. These studies evaluated the agent's ability to inhibit AGE formation and AGE-protein crosslinking compared to that of aminoguanidine (AG) through in vitro and in vivo evaluations (Nakamura et al., 1997; Kochakian et al., 1996). A recent breakthrough in this field is the discovery of a compound, N-phenacylthiazolium bromide (PTB), which selectively cleaves AGE-derived protein crosslinks in vitro and in vivo (Vasan et al., 1996; Ulrich and Zhang, 1997). The pharmacological ability to break irreversible AGE-mediated protein crosslinking offers potential therapeutic use.

It is well documented that early pharmaceutical intervention against the long-term consequences of hyperglycemia-induced crosslinking prevent the development of severe late complications of diabetes. The development of nontoxic and highly effective drugs that completely stop glucose-mediated crosslinking in the tissues and body fluids is a highly desirable goal. The prototype of the pharmaceutical compounds investigated both in vitro and in vivo to intervene with the formation of AGEs on proteins is aminoguanidine (AG), a small hydrazine-like compound (Brownlee et al., 1986). However, a number of other compounds were found to have such an inhibitory effect on AGE formation. Examples are D-lysine. (Sensi et al., 1993), desferrioxamine (Takagi et al., 1995), D-penicillamine (McPherson et al., 1988), thiamine pyrophosphate and pyridoxamine (Booth et al., 1997) which have no structural similarities to aminoguanidme.

Clinical trials of AG as the first drug candidate intended to inhibit AGE formation are in progress (Corbett et al., 1992). A number of hydrazine-like and non-hydrazine compounds have been investigated. So far AG has been found to be the most useful with fewer side effects than other tested compounds of the prior art. AG is also a well known selective inhibitor of nitric oxide (NO) and can also have antioxidant effects (Tilton et al., 1993).

A number of other potential drug candidates to be used as AGE inhibitors have been discovered recently and evaluated both in vitro and in vivo (Nakamura et al., 1997; Soulis et al., 1997). While the success in studies with aminoguanidine and similar compounds is promising, the need to develop additional inhibitors of AGEs continues to exist in order to broaden the availability and the scope of this activity and therapeutic utility.

### SUMMARY OF THE INVENTION

The compounds of the present invention have the following formulas:

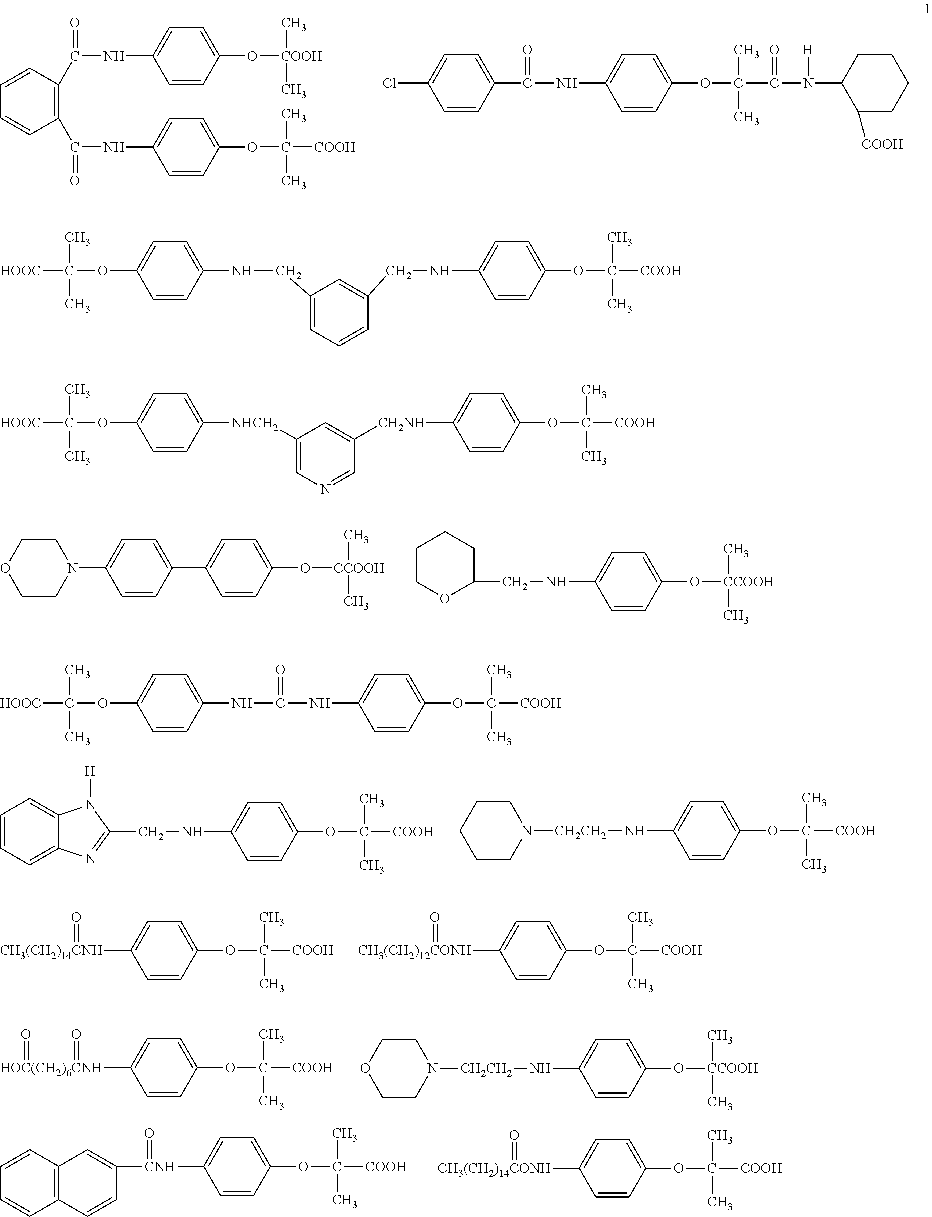

### BRIEF DESCRIPTION OF THE DRAWING

FIG. 1 is an NMR spectra of the compound of Example 5.

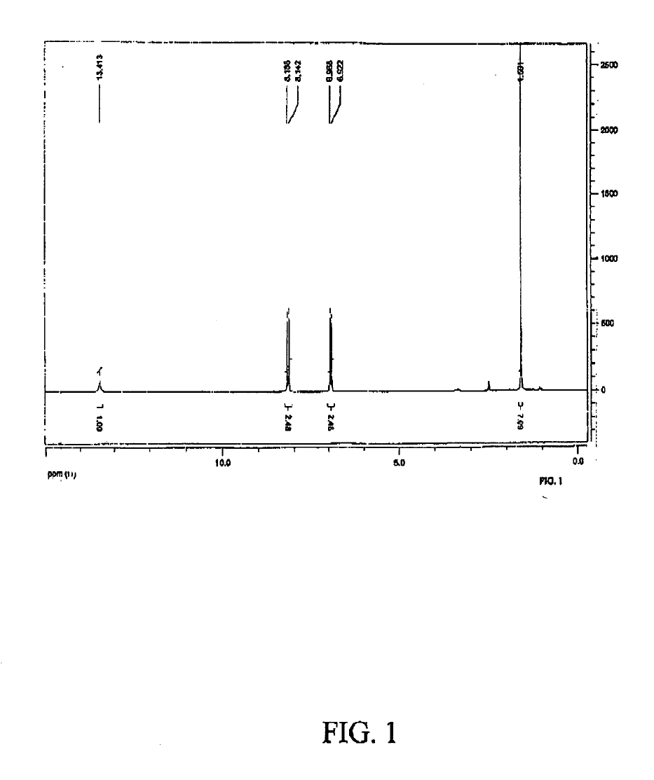

### DETAILED DESCRIPTION OF THE INVENTION

The present inventors have previously reported new classes of compounds which are aryl (and heterocyclic) ureido and aryl (and heterocyclic) carboxamido phenoxyisobutyric acids and also benzoic acid derivatives and related compounds as inhibitors of glycation and AGE formation (Rahbar et al., 1999; Rahbar et al., 2000; Rahbar et al., 2002). See also U.S. Pat. Nos. 5,093,367; 6,072,072; 6,337,350; 6,605,642 and 7,030,133 which are incorporated herein by reference. An elevated concentration of reducing sugars (i.e., glucose) in the blood and in the intracellular environment of an animal, namely a human, typically results in the nonenzymatic formation of glycation and dehydration condensation complexes known as advanced glycation end-products (AGE). These AGE complex products form on free amino groups, on proteins, on lipids and on DNA (Bucala and Cerami, Adv Pharmacol 23:1-34, 1992; Bucala et al., Proc Natl Acad Sci 90:6434-6438, 1993; Bucala et al., Proc Natl. Acad Sci 81:105-109, 1984). This phenomenon is called “browning” or a “Maillard” reaction and was discovered in the last century by the food industry (Maillard, Ann Chim 5:258-317, 1916). The significance of a similar process in biology became evident only after the discovery of the glycosylated hemoglobins and their increased presence in diabetic patients (Rahbar, Clin Chim Acta 20:381-5, 1968; Rahbar et al., Biochem Biophys Res Commun 36:838-43, 1969). A diabetic patient's AGE level increases markedly as a result of sustained high blood sugar levels and often leads to tissue damage through a variety of mechanisms including alteration of tissue protein structure and function, stimulation of cellular responses through AGE specific receptors and/or the generation of reactive oxygen species (ROS) (for a recent review see Boel et al., J Diabetes Complications 9:104-29, 1995). These AGE have been shown to cause complications in patients suffering from various pathological conditions, including, but not limited to, diabetes mellitus, rheumatoid arthritis, Alzheimer's Disease, uremia and in atherosclerosis in persons undergoing hemodialysis.

Advanced glycation end-products bind to cell surface receptors on a variety of cells including, but not limited to, endothelial cells of the microvasculature, monocytes and macrophages, smooth muscle cells, mesengial cells and neurons through a specific receptor for AGEs, termed RAGE. RAGE is a member of the immunoglobulin super family of cell surface molecules. Increased levels of RAGE are expressed in a number of tissues including, but not limited to, aging tissues, diabetic tissues, the vasculature and the kidney. Activation of RAGE has been implicated in a variety of conditions including, but not limited to, acute and chronic inflammation, in certain complications of diabetes, nephropathy, atherosclerosis and retinopathy, Alzheimer's disease, erectile dysfunction and in tumor invasion and metastases. The complications associated with each of these aforementioned pathological conditions places a significant burden on afflicted patients. Moreover, these complications have detrimental effects on society in general. As one example, the global prevalence of diabetes mellitus afflicts millions of individuals resulting in significant increases of morbidity and mortality rates. These increased morbidity and mortality rates, together with the great financial burden of treating diabetic complications, are major incentives to search for and develop medications having the potential of preventing or treating complications of the disease.

The compounds of the present invention inhibit the nonenzymatic formation of glycation and dehydration condensation complexes known as advanced glycation end-products (AGE). In one embodiment of the present invention, a method is provided for administering a medication that inhibits the nonenzymatic formation of glycation and dehydration condensation complexes known as advanced glycation end-products (AGE) to a subject in need thereof, comprising providing at least one medication that inhibits the nonenzymatic formation of AGE complexes; and administering the medication to an patient wherein the nonenzymatic formation of AGE complexes is inhibited.

In another embodiment of the method, the administering step comprises a route of administration selected from the group consisting of oral, sublingual, intravenous, intracardiac, intraspinal, intraosseous, intraarticular, intrasynovial, intracutaneous, subcutaneous, intramuscular, epicutaneous, transdermal, conjunctival, intraocular, intranasal, aural, intrarespiratory, rectal, vaginal and urethral. In another embodiment, the administering step comprises providing the medication on an implantable medical device.

While these medications are typically parameter specific medications, they are efficacious in wound healing, in scar reduction and in the treatment of burns. For example, a compound that inhibits the formation of AGE complexes may be directly applied to in a conventional hydrophilic or oleophilic ointment base, or incorporated within, a medical device (i.e., a wound dressing, patch, etc.) and applied to a patient's skin to aid the wound healing process. MMP9 has been identified in the wound healing process and has also been linked to the inhibitors of AGE

Any method of administering the medication(s) discussed herein is contemplated. While it is understood by one skilled in the art that the method of administration may depend on patient specific factors, the methods of administration include, but are not limited to, generally parenteral and non-parenteral administration. More specifically, the routes of administration include, but are not limited to oral, sublingual, intravenous, intracardiac, intraspinal, intraosseous, intraarticular, intrasynovial, intracutaneous, subcutaneous, intramuscular, epicutaneous, transdermal, conjunctival, intraocular, intranasal, aural, intrarespiratory, rectal, vaginal, urethral, etc. Typically, an oral route of administration is preferred.

Of course, it is understood that the medication will be administered in the appropriate pharmaceutical dosage, depending on the route of administration. For example, an oral dosage form may be administered in at least one of the following pharmaceutical dosage forms: tablet; capsule; solution; syrup; elixir; suspension; magma; gel; and/or powder. A sublingual preparation may be administered in at least one of the following pharmaceutical dosage forms: tablet; troche; and/or lozenge. A parenteral dosage form may be administered in at least one of the following pharmaceutical dosage forms: solution and/or suspension. An epicutaneous/transdermal dosage form may be administered in at least one of the following pharmaceutical dosage forms: ointment; cream; infusion pump; paste; plaster; powder; aerosol; lotion; transdermal patch/disc/solution. A conjunctival dosage form may be administered in at least one of the following pharmaceutical dosage forms: contact lens insert and/or ointment. An intraocular/intraaural dosage form may be administered in at least one of the following pharmaceutical dosage forms: solution and/or suspension. An intranasal dosage form may be administered in at least one of the following pharmaceutical dosage forms: solution; spray; inhalant and/or ointment. An intrarespiratory dosage form may be administered in at least one of the following pharmaceutical dosage forms: aerosol and/or powder. A rectal dosage form may be administered in at least one of the following pharmaceutical dosage forms: solution; ointment and/or suppository. A vaginal dosage form may be administered in at least one of the following pharmaceutical dosage forms: solution; ointment; emulsion foam; tablet; insert/suppository/sponge. A urethral dosage form may be administered in at least one of the following pharmaceutical dosage forms: solution and/or suppository.

The above-noted dosage form(s) may include at least one medication disclosed herein, either alone or in combination with at least one other medication disclosed herein or with a medication not disclosed herein and/or in combination with at least one inert pharmaceutical excipient. These medications may have any release profile including, but not limited to, an immediate release, a controlled release and/or a delayed release profile. If desired, the compounds of the invention may be applied as nanoparticles.

The medical devices include, but are not limited to, implantable medical devices such as, but not limited to, stents (both vascular and urethral), deposition implants (implantable medication releasing device), and/or a medication delivery pumps. Also, contemplated herein are topically applied medical devices including, but not limited to, patches, gauze, wraps, appliques, dressings, coverings, etc. In the case of a medical device, at least one medication may be releasably applied either to at least a portion of the surface of the device, or to a material applied to the surface of a device. Alternatively, at least one medication may be absorbed and/or adsorbed into or onto the device material so long as the medication may be released from the material at a later time.

The medication may be releasably applied to the medical device via any industrially acceptable method, including, but not limited to, spray coating, a waterfall method, heat annealing, etc., however, spray coating is typically preferred. Alternatively, the medical device may include at least one medication, wherein the medication is absorbed and/or adsorbed into or onto the medical device. This may be done by any industrially acceptable method. Also, it is contemplated herein that a medical device may include both at least one medication releasably applied to the medical device itself and/or a coating applied to the device and at least one medication absorbed and/or adsorbed into or onto the medical device itself.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

In the course of screening different classes of organic compounds for investigation of their possible inhibitory effects on advanced glycation endproducts (AGEs), it has been found that most of the phenylureido substituted phenoxy propionic acid derivatives tested have inhibitory effects and several of these compounds were potent inhibitors of AGE-formation at concentrations much lower than an equally inhibiting concentration of aminoguanidine.

The mechanism by which this class of compounds inhibits glycation, AGE-formation, and crosslinking is yet to be known in full. Two major mechanisms, transient-metal-chelation such as copper and iron, and scavenging or trapping of reactive carboynyl intermediates have been proposed to be responsible for AGE-inhibitory function of known AGE-inhibitors.

The mechanism of the inhibitory activities of guanidino compound inhibitors such as two known inhibitors of glycation (aminoguanidine and metformin) is that they are postulated to trap MG and other alpha.-dicarbonyl intermediates of glycation. A most recent study has documented the reaction of metformin with MG and glyoxal (GO), forming guanidino-dicarbonyl adducts further supporting this idea (Ruggiero-Lopez et al., 1999).

Using known assay methods specific for the early (Amadori) and late (post-Amadori) stages of glycation revealed some inhibitors to have greater effects in the early stage and some in the late stage of glycation. However, most of the inhibitor compounds we have investigated are multistage inhibitors. The reaction of reducing sugars with .alpha.- and .epsilon.-amino groups of proteins is not a random process but rather a site specific reaction which depends on the nature and the vicinity of these chemical groups. The future task is to specifically define the site and/or sites of interaction of an inhibitor compound in the complex series of reactions and intermediate substrates, leading to AGE formation and cross-linking.

The development of the novel inhibitors of glycation, AGE formation, and AGE-protein crosslinking expands the existing arsenals of inhibitors of glycation reaction that can find therapeutic applications for the prevention of diabetic complications, as well as the prevention of other diseases associated with increased glycation of proteins or lipids. Furthermore, the availability of these compounds may prove useful as tools to study the cascade of reactions and intermediate substrate in the process of AGE-formation and AGE-protein cross-linking.

The compounds of the invention and their useful compositions utilized in the present invention contain agents capable of reacting with the highly active carbonyl intermediate of an early glycation product thereby preventing those early products from later forming the advanced glycation endproducts or in the alternative as agents for “breaking” or reversing the AGE complexes after they form protein crosslinked compounds which cause protein aging. Doses of 1-2000 mg per day may be used to prevent the formation of AGE complexes or to break AGE complexes depending on the desired effect and the observed response in a patient. The formation of AGE has been linked to several pathologies which may be treated according to the invention including chronic inflammation, neuropathy, atherosclerosis, retinopathy, Alzheimer's disease, erectile dysfunction and diabetes. The compounds of the invention are useful for the treatment of pre-diabetes, Type I and Type II diabetes as well as the prevention and/or treatment of diabetic syndrome or diabetic complications such as elevated lipid levels, elevated cholesterol, retinopathy, kidney damage, circulatory disorders, neuropathy and the like. The compounds of the invention may be used as glycation breakers systemically or topically to reverse glycation and its effects such as facial wrinkles. The compounds of the invention also have activity against rheumatoid arthritis, Wilson's disease, atherosclerosis, neurodegenerative diseases such as Parkinson's or Alzheimer's, multiple sclerosis, neurotoxinemia and metabolic syndrome. An oral dose for these conditions is preferred but other routes of administration may be utilized. An effective amount of an oral dose will be from 1-2000 mg daily preferable given in divided doses. It is presently contemplated that a dose of 250-500 mg daily would be preferred. Other utilities envisioned for the present invention are prevention and treatment of aging of the skin by exerting an anti-aging effect that reduces wrinkles and makes the skin smoother. The compounds may be used as solutions or dispersions in water or a conventional cosmetic cream at a concentration of 0.1 to 10% by weight and used as a cosmetic on the skin to improve the smoothness, texture, decrease the appearance of wrinkles, restore a youthful complexion, provide flexibility and support to skin cells, improve the appearance of the skin by preventing or treating aging of the skin. A particular use is the application of compounds to skin for the purpose of increasing the collagen content which will inhibit or reverse environmental aging effects. The compounds of the invention reduce the amount of MMP9 in the skin which is linked to wound healing and skin repair. Thus, they may be used systemically or topically for scleroderma, acne, psoriasis, inflammation, antioxidant effects or for chelation of metals. They may also be used post laser cosmetic treatment for skin rejuvenation to enhance skin healing and repair post treatment. It is also contemplated that an anti-aging after shave lotion may be formulated with compounds of the invention in a hydro-alcoholic solution at a concentration of 0.01-10 wt % of total composition. For topical use, the compounds may be added to hydrophilic or oleophilic cosmetic bases in amounts of 0.01 to 10% by weight, and preferably 1-5% or they may be applied as a solution, a cream, a dispersion or a gel. For systemic use, the compounds may be administered orally at a dose of 1-2000 mg daily in divided doses. The dose will be adjusted depending on the observed effect using conventional dosing techniques. The compounds also inhibit spoilage of proteins in foodstuffs such as the browning reaction seen in certain fruits. The present agents are also useful in the area of oral hygiene as they prevent discoloration of teeth.

The compounds of the invention also have PPAR activity which is an acronym for peroxisome proliferator activated receptor which are a group of receptor isoforms which exist across biology. They are intimately connected to cellular metabolism (carbohydrate, lipid and protein) and cell differentiation. They are also transcript factors. Several types of PPARs have been identified: alpha, gamma 1, 2 and 3 as well as delta or beta. The alpha form is expressed in liver, kidney, heart, adipose tissues as well as in other tissues. The gamma 1 form is expressed in virtually all tissues including heart, muscle, colon, kidney, pancreas and spleen tissues. The gamma 2 form is expressed mainly in adipose tissue (30 amino acids or longer while gamma 3 is expressed in macrophage, large intestine and white adipose tissue. Delta is expressed in many tissues but mainly in brain, adipose tissue and skin. PPARs dimerize with the retinoid receptor and bind to specific regions on the DNA of the largest genes and when PPAR binds to its ligand, transcription of target genes is increased or decreased depending on the gene. The PPAR activity of the compounds of the invention is a property that confirms that the compounds of the invention are useful as antidiabetic compounds in the manner that the PPAR active compound pioglitazone is useful when administered orally to diabetics. The dose may be from 1 to 2000 mg orally and preferably 250-500 mg orally, daily basis given in divided doses. To aid in the administration, the compound may be combined with a pharmaceutical acceptable diluent or carrier to form a pharmaceutical dosage form. The dosage form can be a liquid, solid, gel for immediate release or controlled release. Common pharmaceutical diluents or carriers are described in the Handbook of Pharmaceutical Excipients, 4^{th} addition, the United States Pharmacopiea, and Remington's Pharmaceutical Science.

### Example 1

### 4-phthaloyldiamidophenoxyisdobutyric acid

The reaction was carried out at by adding to a solution of 4-aminophenoxyisobutyric acid (9.8 gm) (50 mmol) in 150 ml 2N NaOH (about 15 gm NaOH) cooled to near freezing and while stirring gradually adding about 5.05 gm of phthaloyl dichloride by drop wise addition. After overnight stirring at room temp dithionite (about 1 gm) was added and the reaction mixture was filtered. The solution was acidified with acetic acid to give a precipitate which was washed with water and filtered and then air dried. The white solid was dissolved in boiling isopropanol and was allowed to crystallize in a refrigerator. About 50 ml water was added and the mixture was filtered. MP 218-221° C. Yield about 5.2 gm (98%). C₂₈H₂₈N₂O₈; MW 548

### Example 2

### 4-chlorobenzamidophenoxyisobutyrlamidocyclohexl-1-carboxylic acid

4-chlorobenzamidophenoxyisobutyric acid (3.25 gm) (1 mmol) was added to a solution of ethylchloroformate (0.1 ml) and triethyamine (0.15 ml) in 10 ml tetrahydrofuran in an ice salt bath with stirring for ½ hr. Then a solution of 1-aminocyclohexane-1- carboxylic acid (1.46 gm) was added and stirring was continued for 1.5 hr and then the tetrahydrofurqan was evaporated followed by the addition of 20 ml water and the solution was cooled in a freezer (4° C.) and then an excess of 1N NaOH was added (about 20 ml) to make the solution alkaline and to dissolve the solid. Most of the tetrahydrofuran) was evaporated and then the solution was diluted with 20 ml of additional water and acidified with acetic acid to give an immediate crystalline compound which was filtered, washed with water and air dried.

Note: The yield was small and low possibly due to the excess of NaOH.

### Example 3

### 1,3-dibenzylaminophenoxyisobutyric acid

1,3-dichlorodimethylbenzene (1.38 gm) (0.01 mol) was added to 4-aminophenoxyisobutyric acid (2.47 gm) (0.02 mol) and K₂CO₃ (2.76 gm) were dissolved in 25 ml of ethanol (anhydrous, denatured). The mixture was stirred and refluxed for 24 hrs. At the end, water was added and evaporated to remove the ethanol. 1.0 gm dithionite was added, filtered hot and acidified with acetic acid. The compound was solid and the MP 133-135.

### Example 4

### 2,6-pyridyldimethyleneaminophenoxyisobutyric acid)

1.7 gm of 2,6-dimethylaminopyridine (0.01 mol) was added to a mixture of 4-aminophenoxyisobutryic acid (2.9 gm) (2.02 mol) and K₂CO₃ (4.16 gm) (0.03 mol) in ETOH (30 ml, same as above). The mixture was stirred and refluxed overnight and then 30 ml of water according to Example 3. Heating was continued to evaporate the solvents. 1 gm dithionite was added and the solution became discolored (dark orange) due to the presence of monodichloropyridine. The reaction mixture is acidified with acetic acid and a yellow precipitate is formed. This is filtered and washed with water and then air dried giving 1.25 gm The yield was lower than expected, most likely due to water solubility of the product.

### Example 5

### 4-morpholinophenoxyisobutyric acid

A mixture of 0.6 ml dichlorodiethylether MW 149 (0.05 mol) (118 ml) is added to 4-aminophenoxyisobutyric acid 1.95 gm (0.1 mol) and potassium carbonate (MW 1.37) 2.76 gm ((0.2 mol) in 30 ml ETOH (absolute, denatured). This mixture is stirred and refluxed for 24 hrs. After evaporation of most of the ethanol, the solution is acidified with acetic acid and charcoaled to give a white powder which was washed with water and air dried to give 2.06 g. which is 80% of the theoretical yield of 2.25 g. MW 265 C₁₄H₁₉N₁O₄

### Example 6

### 4,2-tetrahydropyranoylaminomethylaminophenoxyisobutyric acid

2-(chloromethyl)tetrahydropyrene (0.64 ml) (0.05 mol) was added to 4-aminophenoxyisobutyric acid (0.975 gm) (0.01 mol) and K₂CO₃ (0.645 gm) (0.05 mol) in 15 ml ethanol and this was stirred and refluxed overnight. The reaction mixture was worked up by adding water and dithionite (0.5 gm) and then filtered, acidified and washed with water and then evaporated. MW 283 C₁₆H₂₃N₁O₄

### Example 7

### 6-methylpthalimidylphenoxyisobutyric acid

6-chloromethylpthalimide (1.7355 gm) (0.01 mol) was added to (1.95 gm) (0.01 mol) of 4-aminophenylsobutryic acid and 1.4 gm of K₂CO₃ in 20 ml of ETOH. This mixture was stirred and refluxed for 45 hours. The brown solution obtained was treated with 1 gm dithionite and evaporated, water is added and the mixture is acidified with citric acid, cooled and filtered. The precipitate was dried to yield about 1 gm. MP 208-212° C. MW 333 C₁₈H₁₉N₃O₄

### Example 8

### 4-adipoylamidodiphenoxyisobutyric acid

4-aminophenoxyisobutryic acid (3.9 gm) (0.02 mol) was dissolved in 5 gm NaOH in 15 ml water while stirring and then cooled on an ice salt bath. Then adipylchloride (1.415 ml) (0.01 mol) was added drop wise and the solution was stirred for 60 hours at room temperature. Then 1 gm dithionite was added and the mixture was filtered. The products was acidified with acetic acid and cooled and filtered. The precipitate was 4.2 gm giving a yield of 78%. MP 215-218° C. MW 532 C₂₆H₃₂N₂O₁₀

### Example 9

### 1-piperidinylethylaminophenoxyisobutyric acid

Piperidinyl chloride-HCl (1.85 gm) (0.01 mol) was added to 4-aminoisobutryic acid (1.95 gm) (0.01 mol) and K₂CO₃ (2.76 gm0 (0.02 mol). This was stirred and refluxed overnight. Because it was not completely solubilized, 3 ml water was added and stirred and refluxed for another 24 hrs. The solution, after cooling, was acidified with acetic acid and it rapidly turned into a crystalline mass. It was cooled, filtered and washed with cold water and then dried. MW 306 C₁₇H₂₆N₂O₃

### Example 10

### 4-lauroylamidophenoxyisobutyric acid

Lauroyl chloride (0.02 mol) is added dropwise to 4-aminophenoxyisobutyric acid (3.9 gm) in 50 ml of water containing NaOH (5.0 gm) with stirring under ice cold conditions and then overnight at room temperature. The color is discharged by dithionite and the solution is acidified with acetic acid to give small crystals. MW 375 C₂₂H₃₅NO₄

### Example 11

### 4-myristoylamidophenoxyisobutyric acid

Myristoyl chloride (0.02 mol) is added dropwise to 4-aminophenoxyisobutyric acid (3.9 gm) in 50 ml of water containing NaOH (5.0 gm) with stirring under ice cold conditions and then overnight at room temperature. The color is discharged by dithionite and the solution is acidified with acetic acid to give small crystals. MW 375 C₂₄H₃₉NO₄

### Example 12

### 4-monosuberoylamidophenoxyisobutyric acid

Suberoyl chloride (0.02 mol) is added dropwise to 4-aminophenoxyisobutyric acid (3.9 gm) in 50 ml of water containing NaOH (5.0 gm) with stirring under ice cold conditions and then overnight at room temperature. The color is discharged by dithionite and the solution is acidified with acetic acid to give small crystals with an 80% yield. MW 325 C₁₈H₂₅NO₆

### Example 13

### 4-morpholinoethyl-1-4-aminophenoxyisobutyric acid

4-aminophenoxyisobutyric acid (1.95 gm) (0.01 mol) was added to 2-chloroethylmorpholine hydrochloride (1.9 gm) (0.01 mol), and K₂CO₃ (2.376 gm) (0.02 mol) which was stirred and refluxed overnight. An insoluble portion remained in the mixture and 30 ml water was added and heating and stirring was continued for another 24 hrs. At this time, 1 gm dithionite was added and the solution was acidified with acetic acid. A light color solid was obtained. MW 307 C₁₆H₂₃N₂O₄

### Example 14

### 4-(2-naphthoylamido)phenoxyisobutyric acid

A solution of 4-aminophenoxyisobutryic acid (0.95 gm) (0.01 mol) and 1.93 gm (0.01 mol) of 2-naphthoylchloride in 15 ml tetrahydrofuran was stirred at room temperature overnight. This was concentrated by heat to remove the tetrahydrofuran (boiling mildly) and then this was acidified with acetic acid. The precipitate turned to a mass which was cooled in a freezer. Water (50 ml) was added and the mixture was filtered and the solid was washed again with water and then air dried. The yield was 3.45 gm. (100%) MP 158-168° C. MW 349 C₂₁H₁₉NO₄

### Example 15

### 4-palmitoylamidophenoxyisobutyric acid

Palmitoyl chloride (0.02 mol) is added dropwise to 4-aminophenoxyisobutyric acid (3.9 gm) in 50 ml of water containing NaOH (5.0 gm) with stirring under ice cold conditions and then overnight at room temperature. The color is discharged by dithionite and the solution is acidified with acetic acid to give small crystals.

### Example 16

### di-adipoylamidophenoxyisobutyric acid

Adipoyl chloride (0.01 mol) is added dropwise to 4-aminophenoxyisobutyric acid (3.9 gm) in 50 ml of water containing NaOH (5.0 gm) with stirring under ice cold conditions and then overnight at room temperature. The color is discharged by dithionite and the solution is acidified with acetic acid to give small crystals with an 80% yield. MW 532 C₂₆H₃₂N₂O₁₀

We claim:

1. A compound of the formula:

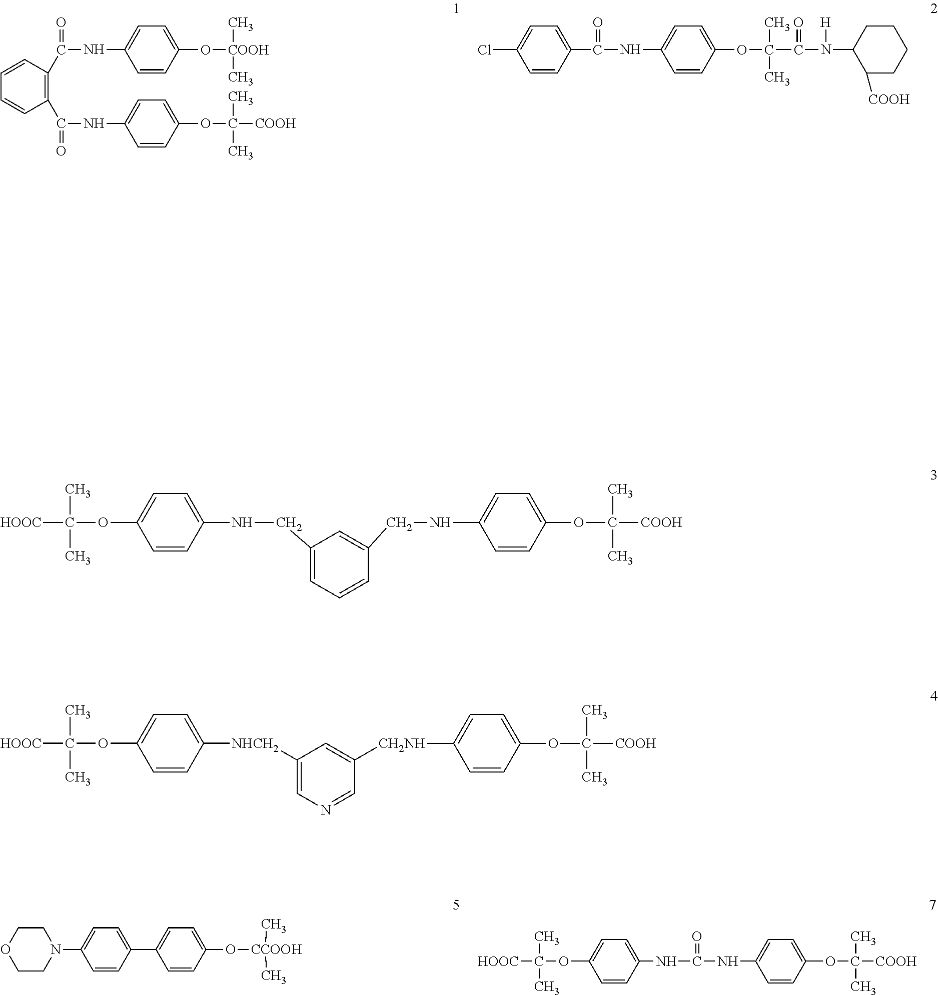

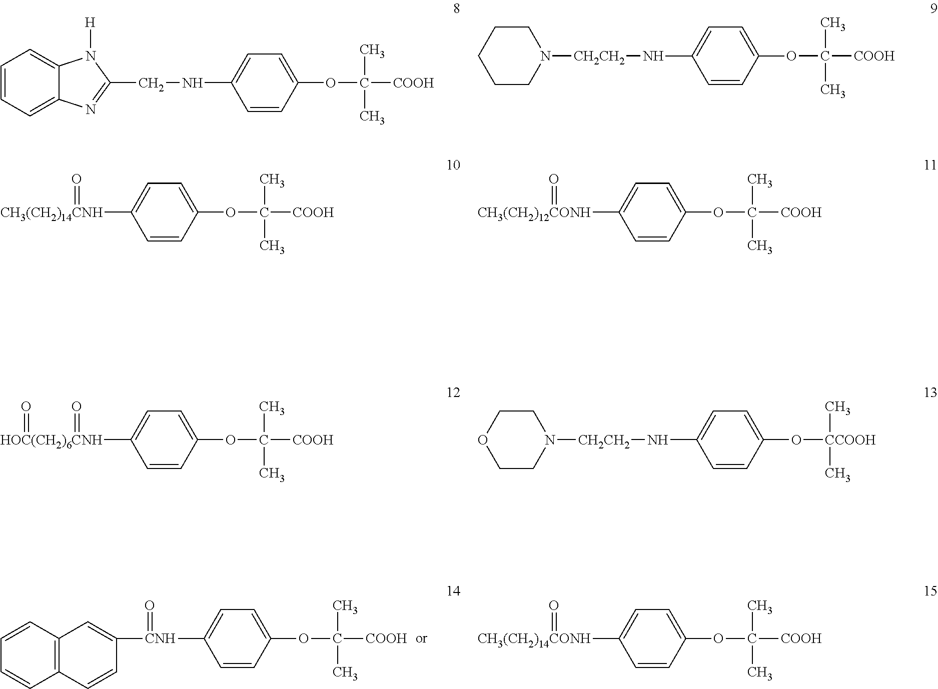

2. A pharmaceutical composition which comprises a compound as defined in claim 1 and a pharmaceutically acceptable diluent.

3. A cosmetic composition which contains a cosmetically effective amount of a compound of claim 1 and a cosmetic carrier composition.

4. A method of improving the appearance of the skin by reducing the appearance of wrinkles and enhancing the smoothness of the skin which comprises applying an effective amount of a compound of claim 1 to the skin of a human.

5. A method of inhibiting formation of glycation end products or protein crosslinking resulting from glycation in a mammal in which it is desired to inhibit formation of glycation endproducts or protein crosslinking, wherein said method comprises administering an amount of a compound as defined in claim 1 in an amount effective to inhibit the formation of glycation end products or protein crosslinking.

6. A method of preventing or treating skin aging which comprises applying to the skin an effective amount of a compound of claim 1 or a pharmaceutically acceptable salt of said compound to said mammal.

7. A method of treating rheumatoid arthritis, Alzheimer's disease, Wilson's disease, atherosclerosis, neurodegenerative diseases and metabolic syndrome wherein said method comprises administering an effective amount of a compound of claim 1 or a pharmaceutically acceptable salt of said compound to said mammal

8. A method of treating skin to modify skin collagen which comprises applying to the skin an effective amount of a compound of claim 1 or a pharmaceutically acceptable salt thereof.

9. A method of preventing aging of the skin which comprises contacting skin with an effective amount of a compound of claim 1 or a pharmaceutically acceptable salt of said compound.

10. A pharmaceutically acceptable salt of a compound of claim 1 and a pharmaceutical carrier.

11. A method of treating Type I diabetes which comprises administering to a Type I diabetic patient an effective amount of a compound of claim 1 or a pharmaceutically acceptable salt of said compound.

12. A method of treating Type II diabetes which comprises administering to a Type II diabetic patient an effective amount of a compound of claim 1 or a pharmaceutically acceptable salt of said compound.

13. A method of breaking glycation end products resulting from glycation in a mammal in which it is desired to break glycation end products wherein said method comprises administering a compound as defined in claim 1 in an amount effective to break glycation end products.

14. A method of breaking end products as defined in claim 13 where the compound is administered topically.

15. A method of inhibiting the formation of glycation end products as defined in claim 5 where the compound is administered topically.
